(19) United States
(12) Patent Application Publication
(10) Pub. No.: US 2015/0283271 A1
(43) Pub. Date: Oct. 8, 2015

(54) METHOD FOR MIMICKING HUMAN CLINICAL TRIAL BY USING NON-HUMAN ANIMALS

(71) Applicant: Crown Bioscience Inc. (Taicang), Taicang (CN)

(72) Inventors: Qixiang Li, Taicang (CN); Sheng Guo, Taicang (CN)

(21) Appl. No.: 14/679,878

(22) Filed: Apr. 6, 2015

(30) Foreign Application Priority Data
Apr. 4, 2014 (CN) 201410135420.0

(51) Int. Cl. A61K 49/00; C07K 16/28

(52) U.S. Cl. CPC A61K 49/0008; C07K 16/2863

(57) ABSTRACT

The present invention provides a method for using non-human animals to mimick human clinical trial comprising: (a) obtaining cells or tissues from n human subjects suffered from a disease, wherein n>1; (b) establishing a control group and a treatment group, wherein i) the control group comprises i control non-human animals, wherein i≧n, wherein cells or tissue from each human subject are grafted to at least one control non-human animal; ii) the treatment group comprises j treatment non-human animals, wherein j≧n, wherein cells or tissue from each human subject are grafted to at least one treatment non-human animal; and iii) each control non-human animal or treatment non-human animal is grafted with cells or tissues from one human subject; (c) administering a first agent to the control group and administering a second agent to the treatment group, wherein the first agent is different from the second agent; (d) obtaining the end point of the control group and the treatment group; and (e) comparing the end point of the control group to the end point of the treatment group.

### RELATED APPLICATION

This application relates to and claims priority benefits from CN Patent Application No. 201410135420.0, filed Apr. 4, 2014, titled “A method for mimicking human clinical trial”, which is hereby incorporated by reference in its entirety.

### FIELD OF THE INVENTION

The present application generally relates to a method for mimicking human clinical trial by using non-human animals.

### BACKGROUND OF THE INVENTION

Patient derived xenografts (PDX or HuPrime®) are believed to best mimic the original human cancers from which the models were derived, and recently become popular for evaluating efficacy of investigational cancer drugs for better predictive power. Mouse clinical trial (MCT), or HuTrial™, is an efficacy trial mimicking phase II clinical trial, where a number of PDXs are testing subjects. Randomized and controlled trial (RCT) with statistical power is a gold standard in the human clinic trial to enable reliable data analysis and conclusion.

However, there are still unmet needs to provide a reliable and efficient method for mimicking human clinical trial by using non-human animals.

### SUMMARY OF THE INVENTION

The present disclosure provides a method for using non-human animals to mimick human clinical trial.

In one aspect, the present invention provides a method for using non-human animals to mimick human clinical trial comprising: (a) obtaining cells or tissues from n human subjects suffered from a disease, wherein n>1; (b) establishing a control group and a treatment group, wherein i) the control group comprises i control non-human animals, wherein i≧n, wherein the cells or tissues from each of the human subjects are grafted to at least one control non-human animal; ii) the treatment group comprises j treatment non-human animals, wherein j≧n, wherein the cells or tissues from each of the human subjects are grafted to at least one treatment non-human animal; and iii) each of the control non-human animals or treatment non-human animals is grafted with cells or tissues from only one of the human subjects; (c) administering a first agent to the control group and administering a second agent to the treatment group, wherein the first agent is different from the second agent; (d) obtaining the end point of the control group and the treatment group; and (e) evaluating the relationship between the first agent and the second agent.

In some embodiments, the disease is a cancer. In some embodiments, the disease is lung, colorectal, gastric, liver, esophageal, ovarian, head and neck, pancreatic, or breast cancer.

In some embodiments, the end point is Progression-Free Survival. In some embodiments, the Progression-Free Survival is the time from administration until tumor volume increases by a pre-determined rate or death. If neither event occurs, then PFS is the time until the last observation day, that is, the censored time. In some embodiments, the pre-determined rate is 50%-90%, 70%-90%, or 70%-80%. In some embodiments, the pre-determined rate is 60%, 70%, 73%, 75%, 80%, 85%, 90% or 95%. In some embodiments, the pre-determined rate is larger than 100%, such as 100%-200%. In some embodiments, the Progression-Free Survival is the time from administration until tumor volume reaches a pre-determined threshold or death. In some embodiments, the pre-determined threshold is 500 mm³-1000 mm³, 600 mm³-800 mm³, 100 mm³-400 mm³, 1000 mm³-1500 mm³ or 1500 mm³-2000 mm³. In some embodiments, the pre-determined threshold is 100 mm³, 200 mm³, 300 mm³, 400 mm³, 500 mm³, 600 mm³, 700 mm³, 800 mm³, 900 mm³, 1000 mm³, 1100 mm³, 1200 mm³, 1300 mm³, 1400 mm³, 1500 mm³, 1600 mm³, 1700 mm³, 1800 mm³, 1900 mm³, 2000 mm³.

In some embodiments, i=j. In some embodiments, i≠j.

In some embodiments, the distribution of the grafted cells or tissues of each of the human subjects is homogeneous in the control group and/or in the treatment group. In some embodiments, the distribution of the grafted cells or tissues of each of the human subjects in the control group is the same with that in the treatment group.

In some embodiments, i and j are pre-determined based on the weighted log-rank statistics. In some embodiments, i and j are pre-determined considering the situation that cells or tissues from each of the human subjects are grafted to at least one non-human animal in control group and treatment group. In some embodiments, the sum of i and j is pre-determined through the formula below:

$$K = \frac{{( {Z}_{1 - α / 2} + {Z}_{1 - β} )}^{2}}{{P}_{1} {P}_{2} {γ}^{2}} ( 1 - ρ ) ,$$

Wherein K is the sum of i and j, α is type I error rate, 1-β is power of statistic, Zₘ is the mth quantile of a standard normal distribution, P₁ is i/K, P₂ is j/K, γ is log (Hazard ratio), and 0<ρ<1.

In some embodiments, the non-human animal is mammal. In some embodiments, the non-human animal is rodent. In some embodiments, the non-human animal is mouse or rat.

In some embodiments, the first agent is a placebo. In some embodiments, the first agent is a vehicle. In some embodiments, the first agent is reference listed drug (RLD) for the disease. In some embodiments, the second agent is a candidate drug for treating the disease. In some embodiments, the second agent is a group of candidate drugs for treating the disease. In some embodiments, the second agent is a candidate anti-cancer agent.

### BRIEF DESCRIPTION OF FIGURES

FIG. 1. The schematic figure for MCT. In the MCT, the cells or tissues from each human subject are grafted to one control non-human animal and to one treatment non-human animal.

FIG. 2. The simulation results for the deviation of ΔT/ΔC from that in the 10:10 design, which was repeated 1000 times to generate distributions of ΔT/ΔC difference. FIG. 2(a) is for high potent drug and FIG. 2(b) is for less potent drug.

FIG. 3. An illustration of the comparison between the needed numbers of samples calculated by different formula (α is 0.05, 1-β is 0.8).

FIG. 4. An illustration of the comparison between the needed numbers of samples calculated by different formula (α is 0.05, 1-β is 0.9).

FIG. 5. An illustration of the relationship between the median PFS of treatment group and that of control group. In the figure, each point means the median PFS of mice grafted with one same PDX in control group or treatment group.

### DETAILED DESCRIPTION OF THE INVENTION

In one aspect, the present invention provides a method for using non-human animals to mimick human clinical trial comprising: (a) obtaining cells or tissues from n human subjects suffered from a disease, wherein n>1; (b) establishing a control group and a treatment group, wherein i) the control group comprises i control non-human animals, wherein i≧n, wherein the cells or tissues from each of the human subjects are grafted to at least one control non-human animal; ii) the treatment group comprises j treatment non-human animals, wherein j≧n, wherein the cells or tissues from each of the human subject are grafted to at least one treatment non-human animal; and iii) each control non-human animal or treatment non-human animal is grafted with the cells or tissues from only one of the human subjects; (c) administering a first agent to the control group and administering a second agent to the treatment group, wherein the first agent is different from the second agent; (d) obtaining the end point of the control group and the treatment group; and (e) evaluating the relationship between the first agent and the second agent.

Non-Human Animal

“Non-human animal” as used herein refers to all vertebrate animals except human, preferably a mammal, such as a dog, a pig, a rabbit, or a rodent (e.g., a mouse, a rat, a hamster, a guinea pig or such like). In some embodiments, the non-human animal is mammal. In some embodiments, the non-human animal is rodent. In some embodiments, the non-human animal is mouse or rat. In some embodiments, the non-human animal is an immuno-compromised non-human animal capable of receiving and supporting a cell or issue from human subject suffered from a disease without mounting a graft-rejection immune response. An “immuno-compromised” animal can either be an immuno-deficient animal which is genetically deprived of T cells, B cells, NK cells or a combination thereof. Alternatively, the non-human animal can be immuno-suppressed by biological or chemical means.

The non-human animal that is grafted with cells or tissues from the human subject suffered from a disease can be named as Patient derived xenografts (PDX or HuPrime®)

Mimick Human Clinical Trial

The method can be use to mimick the human clinical trial and the data of the candidate drug obtained therefrom can be used to predict the effect of the drug during the human clinical trial. When the non-human animal is mouse or rat, the method can be named as the Mouse clinical trial (MCT) or HuTrial™.

The method offers several advantages over human trial: a) the method can be used to design and perform complex tests with significant less regulatory constraints (e.g., using investigational agents at higher dose with more complex combinations, the sample from the same subject can be used repeatedly, and no need for early stop before completion, etc.); b) any single subject (patient) can be assigned to different treatment groups (arms), enabling internal control mechanism and enhancing statistic power; c) the endpoints can be more objectively defined and measured; d) healthy inbred mice can guarantee significantly more consistent drug exposure, avoiding PK and/or other non-tumor related variations; and lastly; e) the potential savings in later clinical trial due to reduction in size and improving success.

In some embodiments, the method can be used to mimick Phase II clinical trial. In other embodiments, the method can be used to mimick Phase I, Phase III or Phase IV clinical trial.

Disease

The term “disease” used herein refers to any type of diseases, which mainly depends on the indication of the candidate agent used in the method. In some embodiments, the disease is a cancer. In some embodiments, the disease is lung, colorectal, gastric, liver, esophageal, ovarian, head and neck, pancreatic, or breast cancer.

Human Subject

The term “human subject” used herein refers to the donor of the cells and/or tissues used in the method. In some embodiments, the human subject does not suffer any disease other than the indication of the candidate agent, or does not suffer any disease that may significantly affect the effect of the candidate agent on the indication thereof, or does not suffer any disease that does not commonly occur in a human subject suffering the indication of the candidate agent. Selection of the human subject may refer to subject recruitment standards of human trial for the corresponding disease, which may be published by drug regulatory agency, e.g., FDA, EMEA and CFDA.

Cells or Tissues

The “cells or tissues” from human subjects of the present invention comprises a disease cell or tissue, which, after being grafted to the animal, can simulate or mimic the human disease or a lesion of the disease.

In some embodiments, the tissues or cells are cancerous tissue or cells. In other embodiments, the cells are blood cells. In certain embodiments, the xenograft cells are Peripheral Blood Mononuclear Cells (PBMC). In certain embodiments, the xenograft cells are lymphocytes or NK cells.

In some embodiments, the tissues or cells are treated before grafting into the non-human animal. The term “treated” when refers to tissue, generally related to any processing methods known in the art, such as washing, homogenization, re-suspension and mixing with a solution (e.g., saline, PBS etc.) or a matrix (e.g., collagen). The term “treated” when refers to cells, includes any processing methods known in the art, such as culture, sub-culture, activating, treatment with an agent, centrifugation, re-suspension, filtration, and mixing with a solution (e.g., saline, PBS etc.) or a matrix (e.g., collagen).

Graft

The graft can be conducted using any suitable methods known in the art, for example, by grafting cells subcutaneously, intraperitoneally, or intravenously through injection; or alternatively, by implanting a fraction of tissue through surgery. In some embodiments, the cells or tissues are cancerous cells, and are grafted to the non-human animal through subcutaneously injection. The methods for grafting have been described in references, including WO2008143795, WO2008140751, U.S. Pat. No. 642,953 or US20140304884, which are hereby incorporated by reference in their entirety.

In some embodiments, once grafted with the cells or tissues, the non-human animal is allowed sufficient time to develop a lesion of the human disease for further use.

The cells and tissues from each human subject are grafted to at least one control non-human animal and at least one treatment non-human animal. In some embodiments, cells or tissues from each human subject are grafted to 2-100 control non-human animals and/or treatment non-human animals, including any the integers within the preceding range or range defined by any two of the integers within the preceding range.

Each control non-human animal or treatment non-human animal is grafted and only grafted with cells or tissues from one human subject, i.e., none of the control non-human animal or treatment non-human animal is grafted cells or tissues from two or more human subjects.

In some embodiments, the number of non-human animals in control group equates to that in treatment group. In some embodiments, the numbers of non-human animals and in treatment group are 2-200, including any the integers within the preceding range or range defined by any two of the integers within the preceding range.

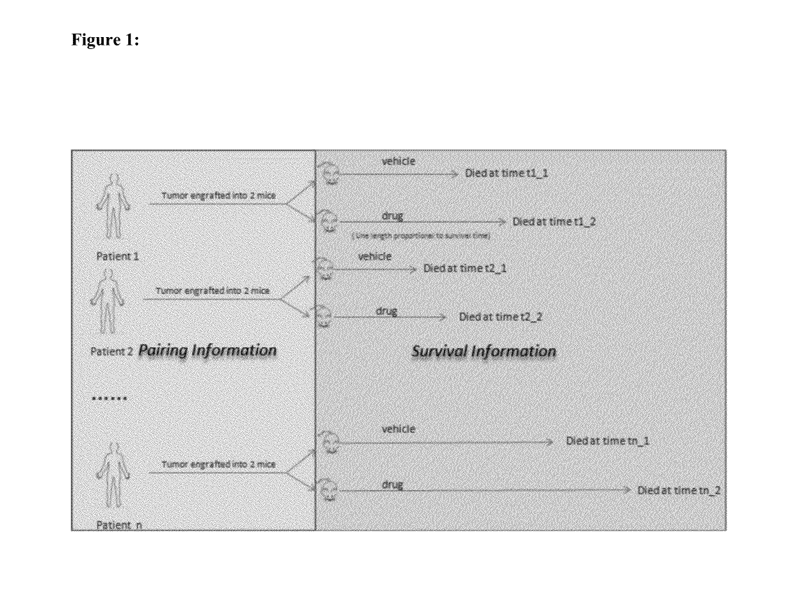

In some embodiments, the distribution of the grafted cells or tissues of each patient is homogeneous in the control group and/or in the treatment group, i.e., the non-human animal grafted with the cells or tissues is homogeneous for the donor human subject in the control group and/or in the treatment group. The term “homogeneous” as used herein refer to, with regard to the cells or tissues from each human subject, grafting number and grafting method is the same between treatment group and control group. In some embodiments, the distribution of the grafted cell or tissue of each patient in the control group is the same with that in the treatment group. In some embodiments, the cells or tissues from each human subject are grafted to one control non-human animal and to one treatment non-human animal as shown in FIG. 1. In some embodiments, the cells or tissues from each human subject are grafted to two control non-human animals and to two treatment non-human animals. In some embodiments, the cells or tissues from each human subject are grafted to greater than 3 control non-human animals and to greater than 3 treatment non-human animals. In some embodiments, the cells or tissues from each human subject are grafted to greater than 3 control non-human animals and to greater than 3 treatment non-human animals.

In some embodiments, the number of the human subjects, the number of control non-human animals in the control group and the number of treatment non-human animals in the treatment group are pre-determined based on the weighted log-rank statistics. Weighted log-rank statistics has been described in the references, including Andersen, P. K., Borgan, Gill, R. D., & Keiding, N. K. (Statistical Models Based on Counting Processes, (1993) New York, Springer-Verlag); Dabrowska, D. M. (Rank tests for matched pair experiments with censored data. (1989) J. Mult. Anal. 28, 88-114); Ronald E. Gangnon & Michael R. Kosorok (Sample-size formula for clustered survival data using weighted log-rank statistics. Tech. Report, Univ. Wisconsin); Fleming, T. R. & Harrington, D. P. (A class of hypothesis tests for one and two sample censored survival data. (1981) Comm. Statist. A 10, 763-94); and Fleming, T. R. & Harrington, D. P. (Counting Processes and Survival Analysis. (1991) New York, Wiley), which are hereby incorporated by reference in their entirety. In some embodiments, the number of the human subjects, the number of control non-human animals in the control group and the number of treatment non-human animals in the treatment group can equate to the number calculated by the weighted log-rank statistics, if there is no drop-out (censored) and all mice reach events. In some embodiments, the number of the human subjects, the number of control non-human animals in the control group and the number of treatment non-human animals in the treatment group can be slightly larger than the number calculated by the weighted log-rank statistics, for example, 10-20%, 20%-30%, 30%-40%, 40%-50%, 50%-100% or 100%-200% more than the calculated number.

In some embodiments, i and j are pre-determined considering the situation that cells or tissue from each human subject are grafted to at least one non-human animal in control group and treatment group. In some embodiments, the sum of the number of control non-human animals in the control group and the number of treatment non-human animals in the treatment group is pre-determined through the formula below:

$$K = \frac{{( {Z}_{1 - α / 2} + {Z}_{1 - β} )}^{2}}{{P}_{1} {P}_{2} {γ}^{2}} ( 1 - ρ ) ,$$

Wherein K is the sum of the number of control non-human animals in the control group and the number of treatment non-human animals in the treatment group, α is type I error rate, 1-β is power of statistic, Zm is the mth quantile of a standard normal distribution, P₁ is i/K, P₂ is j/K, γ is log (hazard ratio), and 0<ρ<1. In some embodiments, ρ can be estimated from historical data, for example, the data obtained from the experiment using the non-human animal grafted by the cells or tissues from human subject suffered same disease and/or using same candidate agent, or can be estimated from early phase data in a trial. In some embodiments, α and β can be freely selected. With fixed α, a functional relationship is established between sample size K and power (1-β). For designs where the cells or tissues from each human subject are grafted to more than one control non-human animals and to more than one treatment non-human animals, or when the distribution of the grafted cell or tissue of each patient is not homogeneous in the control group and/or in the treatment group, the sum of the number of control non-human animals in the control group and the number of treatment non-human animals in the treatment group can be estimated by simulation or extension of the group. The term “power” as used herein refers to the probability that the test correctly rejects the null hypothesis (H₀) when the alternative hypothesis (H₁) is true. It can be equivalently thought of as the probability of correctly accepting the alternative hypothesis (H₁) when it is true—that is, the ability of a test to detect an effect, if the effect actually exists. In some embodiments, the power is 0.5-1, 0.7-1, 0.8-1, 0.9-1, 0.95-1, 0.97-1 or 0.98-1. The term “type I error rate” refers to the probability that the null hypothesis (H₀) is rejected when the null hypothesis (H₀) is true.

In some embodiments, the sum of i and j is no more than the 3, 2, 1.9, 1.8, 1.7, 1.6, 1.5, 1.4, 1.3, 1.2, 1.1, 1 times of the value obtained through the formula below:

$$K = \frac{{( {Z}_{1 - α / 2} + {Z}_{1 - β} )}^{2}}{{P}_{1} {P}_{2} {γ}^{2}} ( 1 - ρ ) .$$

As an example, α is set as 0.01, power of statistic is set as 0.99. According to the MCT design that the amount of the mice in the control group equates to that in the treatment group, P₁ is 1/2 and P₂ is 1/2. Based on the preliminary experiment and the predicted hazard of the tested agent, ρ is 0.3 and the hazard ratio is 0.6. Through the above formula, the K is calculated as 259. In view of that some mice may be censored during the MCT or some mice may never reach the end point during the MCT, the total amount of mice is set as 300. Based on the MCT design, both of the number of the mice in the control group and treatment group are 150. Considering the required number of PDXs (such as 30 PDXs), the number of the mice in the control group and treatment group can be calculated accordingly (such as 5 mice in the control group and 5 mice in the treatment group for each PDX). Therefore, MCT, in which 30 PDXs are used, 5 mice in the control group and 5 mice in the treatment group are grafted by each PDX, is determined. Above MCT can be abbreviated as a MCT of 30 PDXs with 5:5 design.

The First Agent and the Second Agent

“Agent” as used herein refers to any substance, compound or composition suitable for the clinical trial, for example drug, vehicle and placebo. The first agent is different from the second agent. The difference can be different types of agent, different structure of the agents or different dosages of administration for the same agent, which may be selected by a person skilled in the art based on the purpose of the clinical trial. In some embodiments, the first agent is a placebo. In some embodiments, the first agent is a vehicle. In some embodiments, the first agent is reference-listed drug (RLD) for the disease. A person skilled in the art may find the reference-listed drug for the disease in the database of drug administration authority, such as the database of FDA, EMEA or CFDA. In some embodiments, the second agent is a candidate drug for treating the disease. In some embodiments, the second agent is a group of candidate drugs for treating the disease. In some embodiments, the second agent is a candidate anti-cancer agent.

The term “anti-cancer agent” refers to any substance, compound or composition, when administered to a subject, has effect to inhibit, prevent or suppress cancer cell growth, proliferation or metastasis. Examples of the specific anticancer agent may include a c-MET inhibitor, an EGFR inhibitor, an ALK inhibitor, a PDGFR inhibitor, and a c-KIT inhibitor. Examples of the c-MET inhibitor may include PHA-665752, SU11274, XL-880, XL-184, ARQ 197, AMG208, AMG458, CE-355621, and MP470. Example of the EGFR inhibitor may include gefitinib, erlotinib, cetuximab, lapatinib, ZD6474, CL-387785, HKI-272, XL647, PD153035, CI-1033, AEE788, BIBW-2992, EKB-569, and PF-299804. Examples of the ALK inhibitor may include WHI-P154, TAE684, and PF-2341066. Examples of the PDGFR inhibitor may include Gleevec, Desatinib, Valatinib, and Pazopanib. Examples of the c-KIT inhibitor may include Imatinib, Sunitinib, Valatinib, Desatinib, Masitinib, Motesanib, and Pazopanib.

The first agent and/or the second agent will typically be administered to the non-human animal in a dose regimen that provides for the most effective treatment of the disease (from both efficacy and safety perspectives) for which the non-human animal is being treated, as known in the art, and as disclosed, e.g., in International Patent Publication No. WO 01/34574. The first agent and the second agent can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of disease being treated, the type of the first and the second agent being used (e.g., small molecule, antibody, RNAi or antisense construct), and the medical judgement of the prescribing physician as based, e.g., on the results of previous or preliminary experiment. In some embodiments, the manner of administration for the control group is identical to that for treatment group.

The amount of the first agent and/or the second agent administered and the timing of the first agent and/or the second agent administration will depend on the type (e.g., species, gender, age, weight, etc.) and condition of the non-human animal being treated, the severity of the disease or condition being treated, and on the route of administration. For example, the first agent or the second agent can be administered to a patient in doses ranging from 0.001 to 100 mg/kg of body weight per day or per week in single or divided doses, or by continuous infusion (see for example, International Patent Publication No. WO 01/34574). Antibody-based anti-cancer agent, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day. In some embodiments, the amount and timing for non-human animal can be calculated based on the amount and timing for human.

End Point

“End point” as used herein refers to time for occurrence of a disease, symptom, sign or laboratory abnormality that constitutes one of the target outcomes of the trial, or refer to the time for any such disease or sign that strongly motivates the withdrawal of that individual or entity from the trial. In some embodiments, end point is Disease-Free Survival, Progression-Free Survival, Objective Response Rate or Time To Progression. In some embodiments, the Progression-Free Survival refers to the time from administration until tumor volume increases by a pre-determined rate or death. In some embodiments, the pre-determined rate is 50%-90%, 70%-90%, or 70%-80%. In some embodiments, the pre-determined rate is 60%, 70%, 73%, 75%, 80%, 85%, 90% or 95%. In some embodiments, the pre-determined rate is larger than 100%, such as 100%-200%. In some embodiments, the Progression-Free Survival refers to the time from administration until tumor volume reaches a pre-determined threshold or death. In some embodiments, the pre-determined threshold is 500 mm³-1000 mm³, 600 mm³-800 mm³, 100 mm³-400 mm³, 1000 mm³-1500 mm³ or 1500 mm³-2000 mm³. In some embodiments, the pre-determined threshold is 100 mm³, 200 mm³, 300 mm³, 400 mm³, 500 mm³, 600 mm³, 700 mm³, 800 mm³, 900 mm³, 1000 mm³, 1100 mm³, 1200 mm³, 1300 mm³, 1400 mm³, 1500 mm³, 1600 mm³, 1700 mm³, 1800 mm³, 1900 mm³, 2000 mm³.

In some embodiments, the non-human animal is checked so as to obtain the end point. For example, if the end point refers to the time from administration until tumor volume reaches a pre-determined threshold or death, the volume of tumor in the non-human animal is measured. In some embodiments, the non-human animal is checked daily, once in two days, twice weekly, once weekly, once in two weeks, or once monthly. In some embodiments, the frequency of the checking is less than daily, once in two days, twice weekly, once weekly, once in two weeks, or once monthly.

In certain embodiments, the end points of the control group and the treatment are compared to evaluate the relationship between the first agent and the second agent. For example, when the first agent is a vehicle or a placebo and the second agent is a drug candidate drug for treating a disease, the “relationship” may refer to whether the second agent is effective in treating the disease. For another example, when the first agent is a reference listed drug and the second agent is a drug candidate for treating a disease, the “relationship” may refer to whether the second agent is as potent a drug to treat the disease as the first agent.

In some embodiments, the comparison between the end point of the control group and the end point of the treatment group is conducted through statistical software. In some embodiments, the comparison is conducted based on the paring end point. “Paring end point” as used herein refers to the end point obtained from the control non-human animals and treatment non-human animals that are grafted with cells or tissues from the same human subject. In some embodiments, the ratio of mean PFS between the treatment group and the control group is used to measure the efficacy of the second agent. In some embodiments, comparing the end point of the control group to the end point of the treatment group will be conducted on the data obtained after the final dose. In some embodiments, for comparison between two groups, an independent sample t-test will be used. In some embodiments, data will be analyzed using SPSS or R.SAS. In some embodiments, when p<0.05, the data is considered to be statistically significant.

### EXAMPLES

### Example 1

In an MCT of 40 PDXs with 10:10 design (i.e., 40 PDXs is used and, for each of the 40 PDXs, there are 10 mice in the control group and 10 mice in the treatment group), a simulation by randomly drawing n (1≦n≦9) mice from both groups for each PDX was performed. The deviation of ΔT/ΔC from that in the 10:10 design was calculated, which was repeated 1000 times to generate distributions of ΔT/ΔC difference.

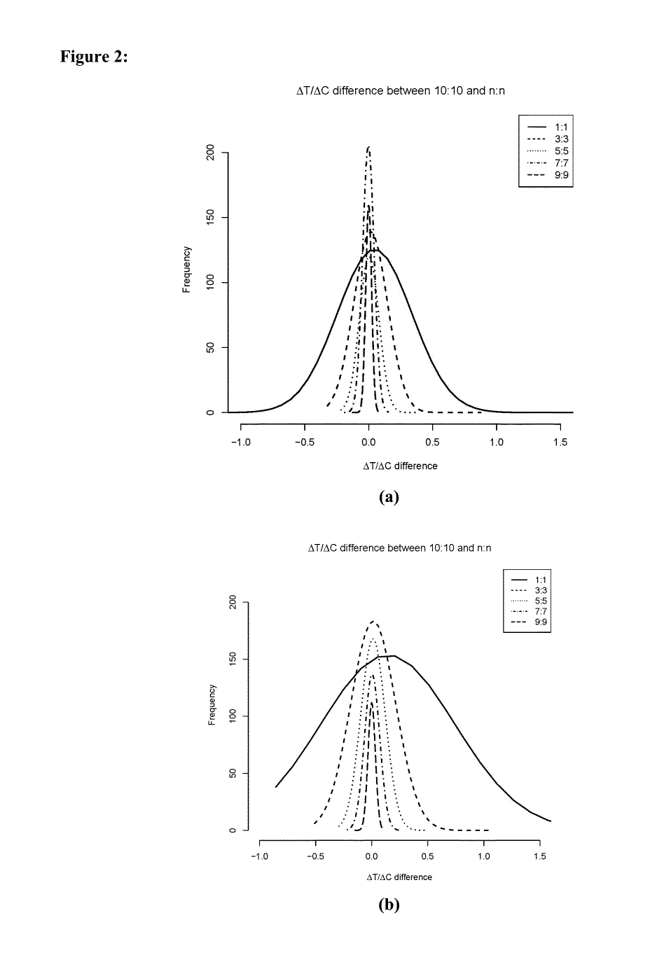

The simulation result is shown in FIG. 2. Based on the MCT, the inventors found that (1) ΔT/ΔC difference has quite large variation in the 1:1 design, and quickly decreases when more mice are added for each PDX model; (2) for the same n:n design, ΔT/ΔC difference is smaller for more potent drugs on the same set of PDX models.

### Example 2

MCT is different from the human clinical trial, since cells or tissues from one human subject may be grafted into multiple non-human animals (e.g., one is grafted into one mice in the control group and is grafted into one mice in the treatment group). Therefore, the end point between non-human animals that is grafted with cells or tissues from the same human subject is usually correlated and cannot be ignored.

However, ordinary log rank test used for human clinical trial ignores the pairing end point (not using the pairing end point), and reduces power. Considering the pairing end point, the sample number can be calculated through a modified formula.

$$\begin{matrix}K = \frac{{( {z}_{1 - α / 2} + {z}_{1 - β} )}^{2}}{{p}_{1} {p}_{2} {γ}^{2}} [ 1 - ρ ] & K = \frac{{( {z}_{1 - α / 2} + {z}_{1 - β} )}^{2}}{{p}_{1} {p}_{2} {γ}^{2}} \\ Paired Survival Analysis & Unpaired Survival Analysis\end{matrix}$$

Wherein K is the sum of the number of control non-human animals in the control group and the number of treatment non-human animals in the treatment group, α is type I error rate, 1-β is power of statistic, Zₘ is the mth quantile of a standard normal distribution, P₁ is i/K, P₂ is j/K, γ is log (hazard ratio), and 0<ρ<1. ρ can be estimated from historical data, for example, the data obtained from the experiment using the non-human animal grafted by the cells or tissues from human subject suffered same disease and/or using same candidate agent, or can be estimated from early phase data in a trial.

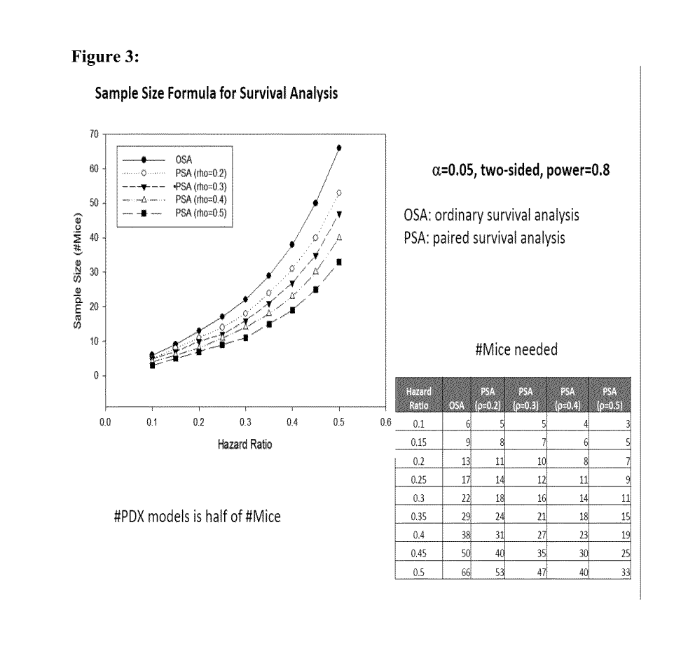

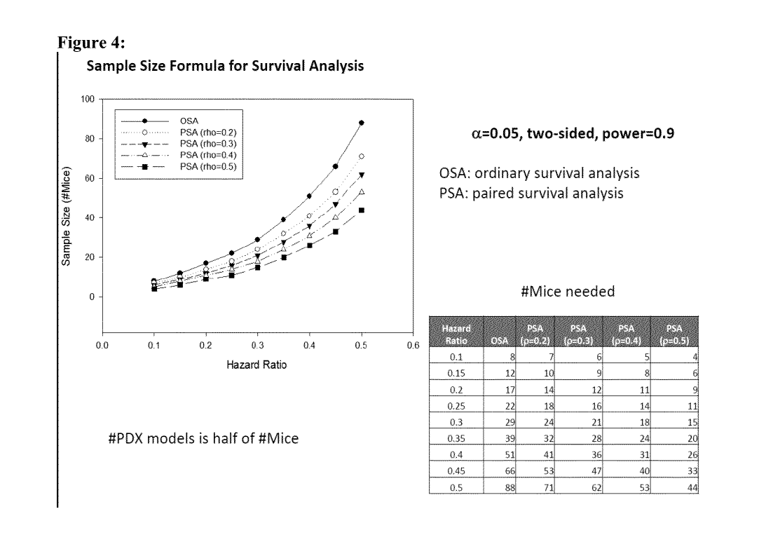

As shown in FIG. 3 and FIG. 4, the number of the samples can be reduced significantly by considering the paring end point.

### Example 3

Based on the modified formula and preliminary experiment, the inventor designs a MCT. In the MCT design, α is set as 0.01, power of statistic is set as 0.99. According to the MCT design that the amount of the mice in the control group equates to that in the treatment group, P₁ is 1/2 and P₂ is 1/2. Based on the preliminary experiment and the predicted hazard of the texted agent, ρ is 0.3 and the hazard ratio is 0.65. Through the above formula, the K is calculated as 363. In view of that some mice may be censored during the MCT or some mice may never reach the end point during the MCT, the total amount of mice is set as 400. Based on the MCT design, both of the number of the mice in the control group and treatment group are 200. Considering 20 PDXs are required, 10 mice in the control group and 10 mice in the treatment group are grafted with each PDX. Therefore, MCT is a MCT of 20 PDXs with 10:10.

In contrast, if paired end point is not considered, i.e., using unpaired survival analysis, the K is calculated as 519. In view of that some mice may be censored during the MCT or some mice may never reach the end point during the MCT, the total amount of mice may be set as 600.

Animals

Species: Mus Musculus; Age: 6-8 weeks; Sex: female; Body Weight (at treatment initiation): 18-20 g; Animal supplier: Beijing HFK Bio-technology Co. Ltd.

Drug

Cetuximab®, an epidermal growth factor receptor (EGFR) inhibitor used for the treatment of metastatic colorectal cancer, metastatic non-small cell lung cancer marked by Bristol-Myers Squibb.

Vehicle

All excipients of Cetuximab without active substance.

Tumor

20 PDXs derived from 20 patients suffered from metastatic non-small cell lung cancer.

Tumor Inoculation

Tumor fragments from stock mice inoculated with selected primary human cancer tissues will be harvested and used for inoculation into BALB/c nude mice. Each mouse will be inoculated subcutaneously at the right flank or in the right mammary fat pad (for some breast models) with primary human tumor fragment (2-4 mm in diameter) for tumor development.

Group Assignment

The tumor volume will be monitored regularly before randomization. When the average tumor size reaches 100-250 mm³ (100-200 mm³ preferred), mice will be randomized into 2 groups and treated as shown in Table 1 for each model.

TABLE 1
Study design of efficacy study

| | N for each | | Dose level | Dose | Dosing |
|---|---|---|---|---|---|
| Group | PDX | Treatment | (mg/kg) | Route | Frequency* |
| 1 | 10 | Vehicle | — | iv | Every day × 49 |
| 2 | 10 | Drug | 40 | iv | Every day × 49 |

*Dosing is stop once the object reaches end point or is censored.

Observation

After tumor inoculation, the animals will be checked daily for morbidity and mortality. At the time of routine monitoring, the animals will be checked for any effects of tumor growth and treatments on normal behavior such as mobility, food and water consumption, body weight gain/loss (twice weekly), eye/hair matting and any other abnormal effect. Death and observed clinical signs will be recorded on the basis of the numbers of animals within each subset.

Tumor size will be measured by caliper twice weekly in two dimensions. The tumor volume will be expressed in mm³ using the formula: TV=0.5 a×b², where a and b are the long and short diameters of the tumor, respectively.

Body weight will be measured twice per week.

End Point

The inventors used Progression-Free Survival (PFS) to evaluate drug efficacy in HuTrial™. We defined PFS as the time from dosing starts until tumor volume reaches 800 cm³. If neither event occurs, then PFS is the time until the last observation day, that is, the censored time. The ratio of mean PFSs between a treatment group and a control group was used to measure drug efficacy.

Termination

Mice were dosed for 49 days before termination.

Under following conditions, the in-life experiment of individual animal or whole groups was terminated, by humane euthanization, prior to death, or before reaching a comatose state: 1) in a continuing deteriorating condition with severe clinical signs of severe distress and/or pain, inaccessible to adequate food or water; 2) Significant Body Weight Loss (emaciated) (>20%); 3) Individual mouse with tumor size exceeding 1500 mm³.

Results

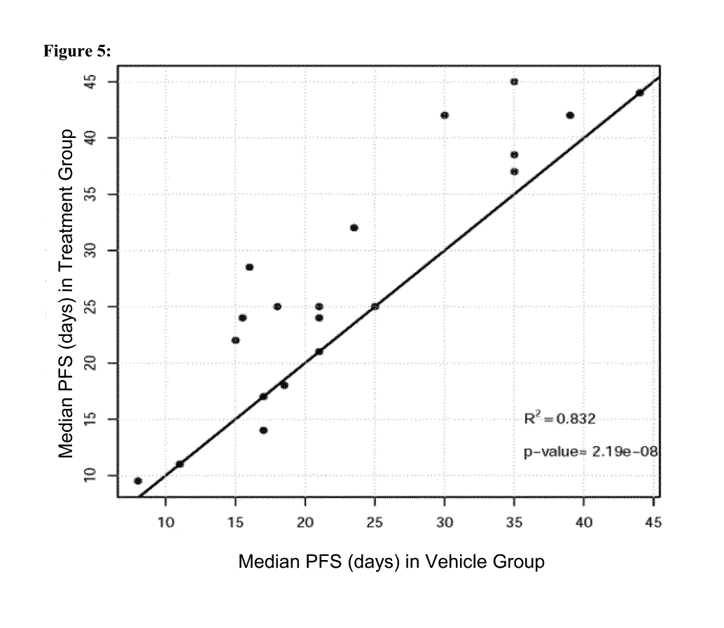

As shown in FIG. 5, the inventors found that the median or mean PFS's in a vehicle group and a treatment groups are highly correlated. Based on the above finding, PFS can be estimated by local linear imputation, which reduces the frequency of the detecting the size of tumor so as to reduce the cost of MCT. Through statistics calculation by SPSS 20, p<0.05, and thus the result is statistically significant.

While the invention has been particularly shown and described with reference to specific embodiments (some of which are preferred embodiments), it should be understood by those having skill in the art that various changes in form and detail may be made therein without departing from the spirit and scope of the present invention as disclosed herein.

What is claimed is:

1. A method for using non-human animals to mimick human clinical trial comprising:
(a) obtaining cells or tissues from n human subjects suffered from a disease, wherein n>1;
(b) establishing a control group and a treatment group, wherein
i. the control group comprises i control non-human animals, wherein i≧n, wherein the cells or tissues from each of the human subjects are grafted to at least one of the control non-human animals;
ii. the treatment group comprises j treatment non-human animals, wherein j≧n, wherein the cells or tissues from each of the human subjects are grafted to at least one of the treatment non-human animals; and
iii. each of the control non-human animals or treatment non-human animals is grafted with the cells or tissues from only one of the human subjects;
(c) administering a first agent to the control group and a second agent to the treatment group, wherein the first agent is different from the second agent;
(d) obtaining the end point of the control group and the treatment group; and
(e) evaluating the relationship between the first agent and the second agent.

2. The method of claim 1, wherein the disease is a cancer.

3. The method of claim 1, wherein the end point is Progression-Free Survival.

4. The method of claim 1, wherein the Progression-Free Survival is the time from administration until tumor volume increases by a pre-determined rate or death.

5. The method of claim 1, wherein the pre-determined rate is selected a group consisting of 50%-90%, 70%-90% and 73%.

6. The method of claim 1, wherein the Progression-Free Survival is the time from administration until tumor volume reaches a pre-determined threshold or death.

7. The method of claim 1, wherein the pre-determined threshold is selected a group consisting of 500 mm³-1000 mm³, 600 mm³-800 mm³, 100 mm³-400 mm³, 1000 mm³-1500 mm³ and 1500 mm³-2000 mm³.

8. The method of claim 1, wherein i=j.

9. The method of claim 1, wherein the distribution of the grafted cells or tissues of each of the human subjects is homogeneous in the control group and/or in the treatment group.

10. The method of claim 1, wherein the distribution of the grafted cells or tissues of each the human subjects in the control group is the same with that in the treatment group.

11. The method of claim 1, wherein i and j are pre-determined based on the weighted log-rank statistics.

12. The method of claim 1, wherein i and j are pre-determined considering the situation that cells or tissues from each of the human subjects are grafted to at least one non-human animal in control group and treatment group.

13. The method of claim 1, wherein the sum of i and j is pre-determined through the formula below:
$$K = \frac{{( {Z}_{1 - α / 2} + {Z}_{1 - β} )}^{2}}{{P}_{1} {P}_{2} {γ}^{2}} ( 1 - ρ ) ,$$
Wherein K is the sum of i and j, α is type I error rate, 1-β is power of statistic, Zm is the mth quantile of a standard normal distribution, P1 is i/K, P2 is j/K, γ is log (Hazard ratio), and 0<ρ<1.

14. The method of claim 1, wherein the non-human animal is mammal.

15. The method of claim 1, wherein the non-human animal is rodent.

16. The method of claim 1, wherein the non-human animal is mouse or rat.

17. The method of claim 1, wherein the first agent is a placebo.
